(19) United States
(12) Patent Application Publication
(10) Pub. No.: US 2011/0230886 A1
(43) Pub. Date: Sep. 22, 2011

(54) DRILL ASSEMBLY AND SYSTEM AND METHOD FOR FORMING A PILOT HOLE

(76) Inventors: Ramon B. Gustilo, Eden Prairie, MN (US); Patrick R. Cornelle, Minneapolis, MN (US); Jude L. Sasing, Quezon City (PH); Jeremy J. Ling, St. Paul, MN (US)

(21) Appl. No.: 13/130,452

(22) PCT Filed: Nov. 20, 2009
(86) PCT No.: PCT/US09/06214

§ 371 Date: May 20, 2011

Related U.S. Application Data
(60) Provisional application No. 61/199,894, filed on Nov. 21, 2008.

(51) Int. Cl. A61B 17/16; A61B 17/58

(52) U.S. Cl. 606/80; 606/96

(57) ABSTRACT

A drill assembly comprises a motor guide tube, an adaptor extending from a distal end of the motor guide tube, an elongate drill guide tube extending from a distal end of the adaptor, a retractable guide tube including an arcuate distal end slideably disposed within a channel of the drill guide tube, an actuation lever operably coupled to the retractable guide tube and extending from an outer surface of the adaptor, a flexible drill cable slideably disposed within the retractable guide tube, a drill motor operably coupled to the flexible drill cable, and a push-pull cable extending between the drill motor at a distal end and a liner stage motor inside a control box at a proximal end. A distal end of the flexible drill cable is structured to be advanced through the arcuate distal end of the retractable guide tube, and the linear stage motor is operable to advance and retract the push-pull cable and the drill motor.

### TECHNICAL FIELD

The present invention relates to flexible cutting tools and more particularly to a drill assembly system and method for drilling a pilot hole from an interior channel of an intramedullary rod or nail.

### BACKGROUND OF THE INVENTION

Intramedullary rods are commonly used in orthopedic surgery for breaks in the long bones of the extremities, such as the femur and tibia. These rods are used to align and stabilize fractures or breaks of bones and to maintain the bone fragments in their proper alignment relative to each other during the healing process. In addition, intramedullary rods can provide strength to the bone during the convalescence of the patient. One common surgical rod implantation procedure involves drilling the bone marrow canal of the fractured bone from a proximal to a distal end of the bone and inserting an intramedullary rod into this evacuated space. In order to maintain the intramedullary rod in the proper relationship relative to the bone fragments, it is often desirable to insert bone screws or other fasteners through the distal and proximal portions of the intramedullary rod and one or both fragments of the bone. Such a fixation of the rod can make the construct more stable, prevent rotation of the rod within the bone, and prevent longitudinal movement of the bone relative to the intramedullary rod.

In order to fix the rod to the bone, intramedullary rods are commonly provided with at least one aperture through each of their proximal and distal end portions for receiving screws or fasteners of various configurations. To insert such screws, the objective is to drill holes through the tissue and bone in proper alignment with the holes in the intramedullary rod, and to insert the screws through the holes to lock the intramedullary rod in place. Locking the rod near its proximal end (near its point of insertion) is usually accomplished with the help of a jig that helps to locate the proximal hole(s) in the rod. In this proximal region, a relatively short-armed aiming device can be attached to the intramedullary rod for reference. A drill can then be passed through the bone and a proximal hole. This technique is relatively straightforward due to the short distance between the accessible proximal end of the rod and the proximal holes in the rod. However, due to the distance between the proximal end of the rod and the point where the holes must be drilled in the bone at the distal end of the rod, it can be difficult to register the drilled hole(s) with the holes in the distal end of the rod. This is particularly true in cases where rod deformation occurred during insertion of the rod into the intramedullary cavity. It can therefore be difficult to successfully align transverse screws with the distal hole(s) for insertion through the bone wall.

Two primary reasons for failure in distal locking of the intramedullary rod to the bone include using an incorrect entry point on the bone and having the wrong orientation of the drill. If either of these two factors exists, the drill may not go through the nail hole. An inaccurate entry point also compounds the problem if the rounded end of the drill bit is slightly out of position, thereby weakening the bone and sometimes making it difficult to find a strong point in the bone in which to place the correct drill hole. Inaccurate distal locking can lead to premature failure with breakage of the nail through the nail hole, breakage of the screw, or the breaking of the drill bit within the bone. In addition, if the distal end of the rod is not properly secured, bone misalignment and/or improper healing of the bone may occur.

One known technique for locating a distal hole in an intramedullary rod is with an x-ray imaging technique in combination with a free hand drilling technique. This technique involves watching a fluoroscopic image intensifier to accomplish distal targeting. However, this technique is difficult to use and adds the additional risk of exposing the patient and surgical team to excessive radiation. Even if protective gloves and clothing are utilized, there can still be risks involved with radiation exposure. This can particularly occur in cases where locating the hole(s) in the rod requires multiple attempts. In addition, if the correct alignment of the components is not obtained on the first attempt, multiple perforations of the bone can be required, which can be detrimental to recovery of the patient and the strength of the bone in this area.

Alternative techniques for locating the distal holes in an implanted intramedullary rod have been proposed. However, such methods are often relatively complex and can require additional electronic equipment and visual displays for operation. Such techniques may require special training and/or machine operators, and can be relatively expensive. These techniques can thus be undesirable in the crowded space of a surgical suite, particularly when it is desirable to minimize the amount of equipment and personnel involved in the surgery. Thus, there is a continued need for additional surgical drilling tools and methods for locating the distal holes in an implanted intramedullary rod. There is a further need to provide such tools and methods that allow for easy and accurate insertion of screws through the bone and rod at the distal rod end. There is even a further need to provide such tools and methods in a relatively economical manner that includes disposable and reusable components.

### BRIEF SUMMARY OF THE INVENTION

The present invention is directed to an orthopedic device for facilitating the fixation of a distal portion of a device to a bone. In one exemplary embodiment, the orthopedic device can facilitate accurate distal fixation of an intramedullary rod within a fractured or damaged bone where the distal fixation area is difficult to locate. Because the devices and methods of the invention do not typically require the use of x-rays or other scanning techniques, the amount of radiation to which the physician is exposed during the distal fixation process is greatly reduced or eliminated. In addition, the process of accurately drilling through the bone and locating corresponding holes in the intramedullary rod is much faster than conventional methods that rely primarily on radiation screening and trial-and-error techniques for proper screw placement.

The orthopedic device of the invention may be referred to as a bone drill or drill. This drill is used for accurately locating the distal holes of an implanted intramedullary rod from within the rod. In particular, this device can drill outwardly from inside the intermedullary rod through the thickness of the bone, and to the outside of the bone. By drilling from inside the rod and using the distal holes to locate the drilling site, the holes drilled through the bone are accurately aligned with the distal holes in the rod. This enables the operator to easily and accurately place the screws in their desired locations to fix the distal portion of the intramedullary rod to the broken bone. One embodiment of the device includes a flexible Nitinol cable that extends from an elongated member and functions as the rotating “drill bit” in the drilling process. The device can be used to determine the location of holes in the distal portion of an implanted rod and to drill a pilot hole through the bone adjacent to the distal rod end to locate an accurate point for screw entry. In alternative embodiments the drill cable may be non rotating and may instead include a means for delivering energy to the distal end of the cable such that a pilot hole may be formed in the bone via ablation.

It is a further advantage of the bone drills of the present invention to provide both components that are reusable and components that are disposable. In particular, the present invention provides a component that is referred to as a disposable drilling assembly, which includes a number of parts that would be difficult, economically impractical, or impossible to sanitize for reuse. However, other components of the systems that are used in combination with the disposable drilling assembly can be reused after proper sanitation. This can help to keep the costs of providing instruments more reasonable.

### BRIEF DESCRIPTION OF THE DRAWINGS

The present invention will be further explained with reference to the appended Figures, wherein like structure is referred to by like numerals throughout the several views, and wherein:

FIG. 1 is a cross-sectional front view of a fractured femur bone of a human with an intramedullary rod inserted into its intramedullary cavity;

FIG. 2 is an isometric view of the complete bone drilling assembly of the invention connected to an intramedullary rod;

FIG. 3 is a perspective view of the drill control system for use with the drilling devices of the invention showing the front panel;

FIG. 4 is a perspective view of the same control system in FIG. 3, but showing the rear panel;

FIG. 5 is a perspective view of the internal components of the control system of FIGS. 3 and 4;

FIG. 6 is a front view of a hand control that is attachable to the control box of FIGS. 3 to 5;

FIG. 7 is a perspective \view of a drill motor assembly;

FIG. 8 is a perspective view of a disposable drilling assembly of the invention with a cut away view inside the guide tube;

FIG. 9 is a cross section of the view of the assembly in FIG. 8;

FIG. 10 is a side view of the drill guide assembly;

FIG. 11 is an external view of the drill assembly identified in FIGS. 8 and 9;

FIG. 12 is a cross section of the view of the assembly in FIG. 11;

FIG. 13 is an external view of an intramedullary nail or rod interface assembly with an attached disposable drilling assembly and incision guide assembly connected to an intramedullary rod in a femur;

FIG. 14 is a cross section view of the assembly in FIG. 13 but with the disposable drilling and incision guide assemblies removed;

FIG. 15 is a perspective view showing a holster that is used to hold the hand control assembly and the disposable drilling assembly of the invention (both shown as well);

FIG. 16 is an external view a suction rod assembly;

FIG. 17 is a top view of the jig interface mated with a disposable drilling assembly; and

FIG. 18 is a cross section view of an intramedullary rod in a bone with a chase back pin inserted into a pilot hole made by the disposable drilling assembly.

### DETAILED DESCRIPTION

The embodiments of the present invention described below are not intended to be exhaustive or to limit the invention to the precise forms disclosed in the following detailed description. Rather the embodiments are chosen and described so that others skilled in the art may appreciate and understand the principles and practices of the present invention.

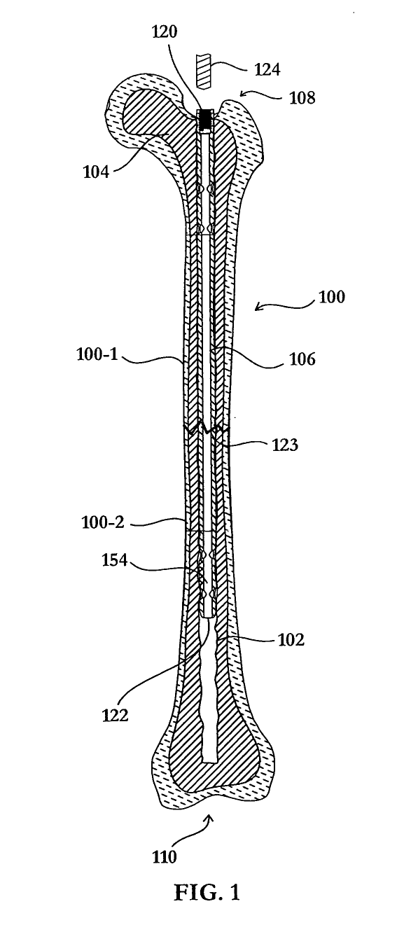

Referring now to the Figures, wherein the components are labeled with like numerals throughout the several Figures, and initially to FIG. 1, a cross-sectional view of two portions of a broken femur 100-1 and 100-2 are illustrated. While the break is generally illustrated as a clean fracture 123 of the bone into two portions, it is possible that the femur 100 could instead be fractured into a number of smaller bone fragments or damaged in some other way. Thus, it is understood that the devices and methods described herein for two bone pieces can also apply to three or more bone pieces or fragments or even a cracked bone that has not separated into multiple pieces. The femur 100 includes cancellous tissue 104 and an intramedullary cavity 102 that extends along a portion of the length of the femur 100 within the tissue 104. The intramedullary cavity 102 is a generally open area in the femur that is filled or partially filled with bone marrow. In order to prepare a bone such as the femur 100 for insertion of an intramedullary rod 106 therein, the intramedullary canal 102 can be aspirated and/or lavaged to remove some or all of the marrow and/or loose materials therein.

FIG. 1 also illustrates the femur 100 with its two fractured portions 100-1 and 100-2 aligned and brought into contact with each other, and an exemplary intramedullary rod 106 inserted within the intramedullary cavity 102. This intramedullary rod 106 includes a cannula or bore 154 that runs generally from a proximal end 120 of the rod 106 to its distal end 122. In order to access the intramedullary cavity 102, a hole can be drilled or reamed in the cortical layer of the bone at its proximal end 108 with a drill shown as 124. The intramedullary rod 106 can then be inserted into the bone through this hole and pushed or hammered downward through the cancellous tissue 104 of the femur 100 and through the intramedullary channel toward the distal end 110 of the femur 100. The intramedullary rod 106 can continue to be tamped or pressed downwardly until the distal end 122 of the rod 106 is in its desired position relative to the distal end 110 of the femur 100 and the proximal end 120 of the rod 106 is in its desired position relative to the proximal end 108 of the femur.

The above discussion of the insertion of an intramedullary rod into a long bone, such as a femur, is intended as one exemplary procedure for such a rod implantation. A number of alternative procedures can be used, along with a number of alternative intramedullary rod designs. However, in accordance with the invention, the intramedullary rod will generally include a central opening at its proximal end, a bore through its center that runs along at least a portion of the length of the rod, and at least one distal hole spaced from the proximal end, such as near the distal end of the rod. It is desirable in many embodiments that the intramedullary rod also has at least one proximal screw hole at its proximal end. In such embodiments, it is further desirable that the proximal and distal holes are spaced from each other by a distance that allows the rod 106 to be sufficiently fixed to the multiple bone segments.

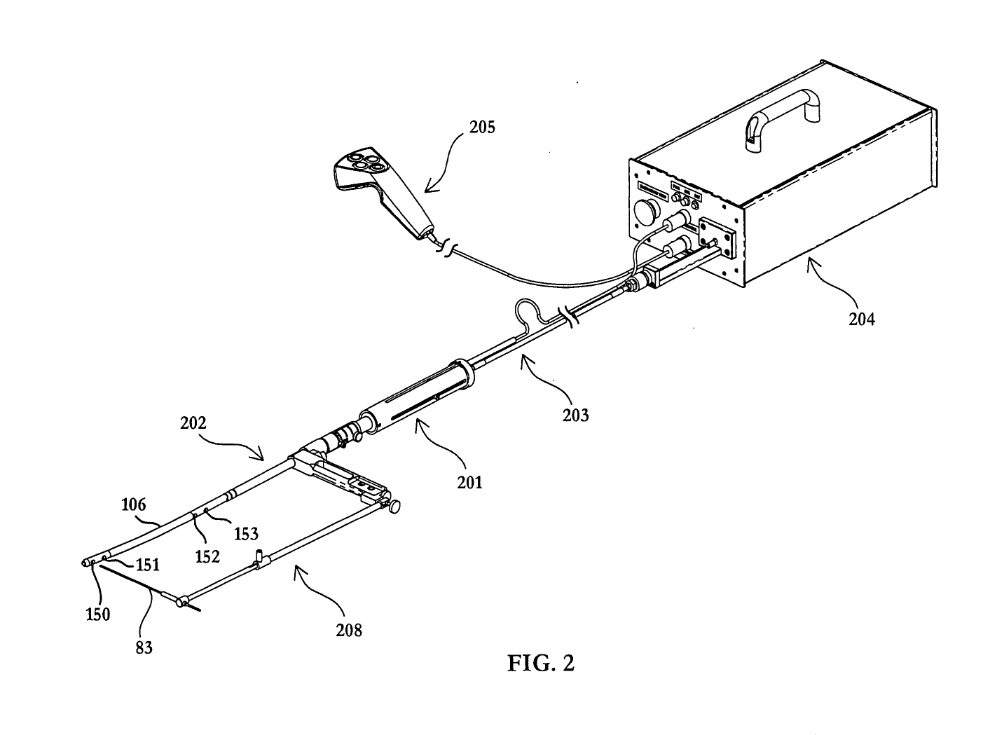

FIG. 2 illustrates a disposable drill assembly 201 as attached to a femoral attachment jig assembly 202 as attached to an intramedullary rod 106, in accordance with the invention. This disposable drill assembly 201 includes a number of features that will be discussed in detail below. The drill assembly 201 is further attachable to a drill motor assembly 203, drill control box system 204, hand control assembly 205, and other components, as will be described in further detail below. Drilling devices and methods for drilling a bone using a flexible cable drill are described in U.S. Patent Application Pub. No. 2008/0114365 (Sasing, et al.), which is incorporated herein by reference in its entirety.

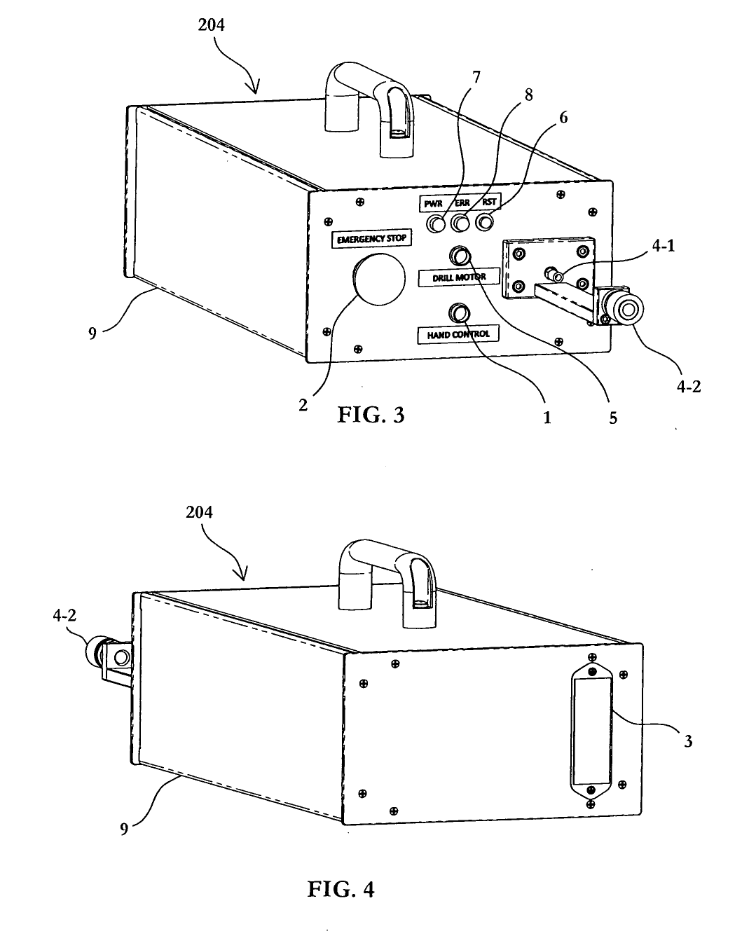

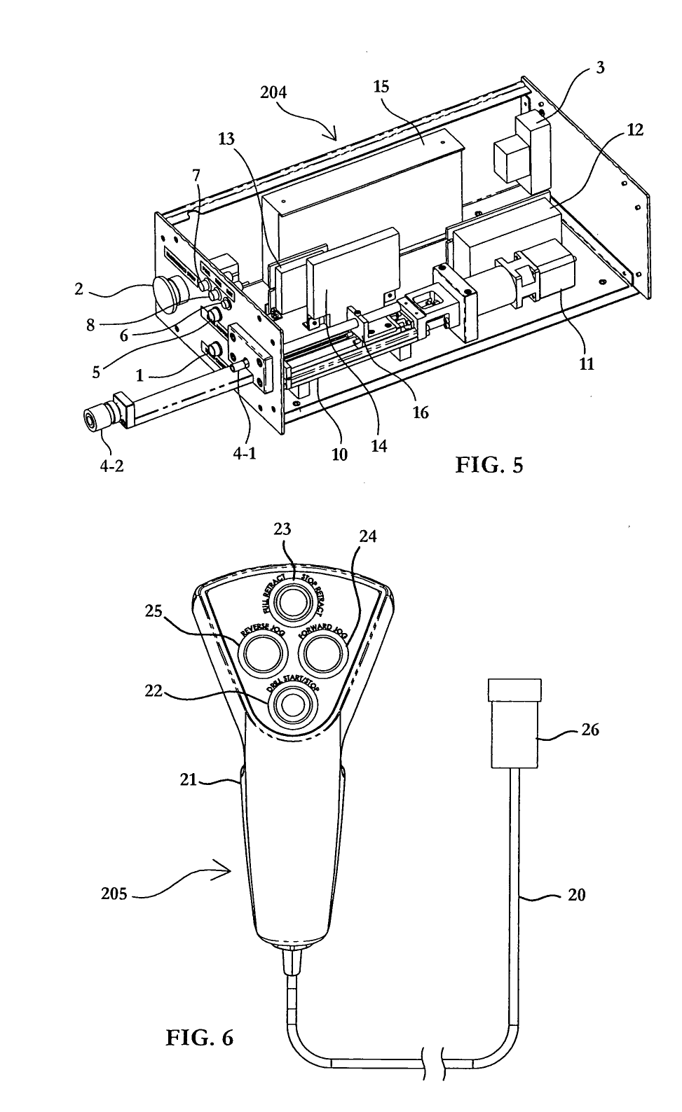

Referring to FIGS. 3 and 4, a drill control box system 204 for use in controlling the bone drill includes a hand control connector 1, an emergency stop switch 2, a power entry module with integrated on/off switch 3, a push/pull inner fitting 4-1, a push-pull outer fitting 4-2, a drill motor connector 5, a reset button 6, a power indicator light 7, and an error indicator light 8. An enclosure 9 provides a mounting surface for these components and protects the internal components shown in FIG. 5. When in use, the control system 204 can be placed adjacent to but outside a sterile field, such as on a secure stand or table. A power cord (not illustrated) can then be connected to the power entry module 3 located at the rear of the box, and then the power switch of the power entry module 3 can be toggled to the “on” position. The control box may include more, less, or different features than described and shown relative to FIGS. 3 and 4. In addition, the system may include more than one control system, such as when it is desired to control certain operations from separate locations.

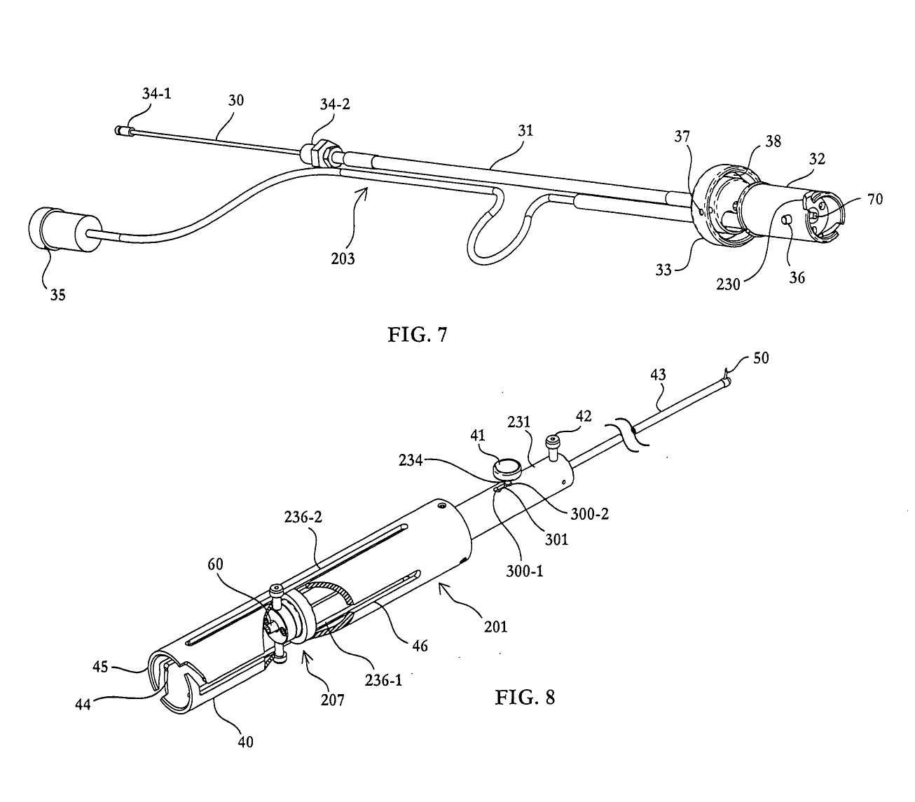

FIG. 5 shows a perspective view of inside of the control system with one side panel and the top cover removed. For improved visualization non-critical details such as internal cabling within the box connecting different components together is not illustrated. The main components inside control system 204 include a linear stage 10 attached drive motor 11 and associated drive controller 12, a drill motor controller 13, a main control board 14, and a power supply 15. The push/pull inner fitting 4-1 is coupled to the linear stage 10 with bracket 16. As will be described later, the linear stage 10 and connected push/pull inner fitting 4-1 is used to advance and retract the drill motor 32 (FIG. 7) when it is connected to the disposable drill assembly 201 with the drill motor assembly 203. The linear stage motor 11 is controlled by drive controller 12, which in turn is controlled by pre-programmed circuitry associated with the main control board 14. In the same manner, the drill motor controller 13 is controlled by the main control board 14. Power supply 15 provides all the necessary power for the components of the control box system 204 and any associated and attached components.

A hand control assembly 205 is shown in FIG. 6 and is provided for attachment to the control system 204. The hand control assembly 205 is the portion of the device used to manage the operation of the drilling procedure. The hand control assembly 205 can be placed on a holster or other device, if desired, and a cord 20 from the hand control 21 can be plugged into the front of the control system 204, in this case into connector 1 shown in FIGS. 3 and 5. This plug-in location can be labeled on the control box as “hand control”, for example. The hand control may include a number of different buttons or control features, such as a drill start/stop control 22, a full retract control 23, a jog forward control 24, a jog backward control 25, and a hand control connector 26. Different means can be used to identify the function of the buttons to the user. The hand control 21 may have labeling as illustrated in FIG. 6 adjacent each button to identify the function of each button, and/or each button may be colored or have colored illumination that corresponds to a specific function. The hand control may include more, less, or different buttons or control features, depending on the drill functions that are to be controlled or managed by the operator. This hand control can be covered with a disposable drape and can be wiped down after its use.

A drill motor assembly 203 of the type illustrated in FIG. 7 is attachable to the front of the control system 204 at location 4-1, 4-2 and 5 shown in FIGS. 3 and 5. This attachment location can be labeled on the control box 9 as “drill motor”, for example. The drill motor assembly 203 may include an inner push/pull cable 30, an outer push/pull guide 31 connected to motor guide-tube cap 33, a drill motor 32, an inner control system attachment 34-1, an outer control box attachment 34-2, and a drill motor connector 35. The drill motor may include more, less, or different cables, housing, guides, and/or other components, depending on the drill control that is desired for the surgical procedure. The drill motor assembly 203 is a transition component between the control box system 204 in the nonsterile field and the parts connected to the control box system that reside in the sterile field. It may also be considered to be a single-use type of component in that it can be designed to be discarded after it is used for one surgical procedure. However, all or parts of the drill motor assembly 203 can also be made to be sterilized for reuse.

Referring to FIGS. 3 and 7, in one exemplary embodiment of the drill motor assembly 203, an inner cable 30 of the drill motor assembly 203 is threaded through the outer fitting 4-2 of the control box system 204, and then the outer connector 34-2 is snapped or connected to the outer fitting 4-2. The inner cable 30 of the drill motor assembly 203 can then be snapped or connected to the inner fitting 4-1 of the control box system 204. The drill motor connector 35 is connected to associated control box system connector 5. The control box system 204 and components will then be configured as is generally shown in FIG. 2.

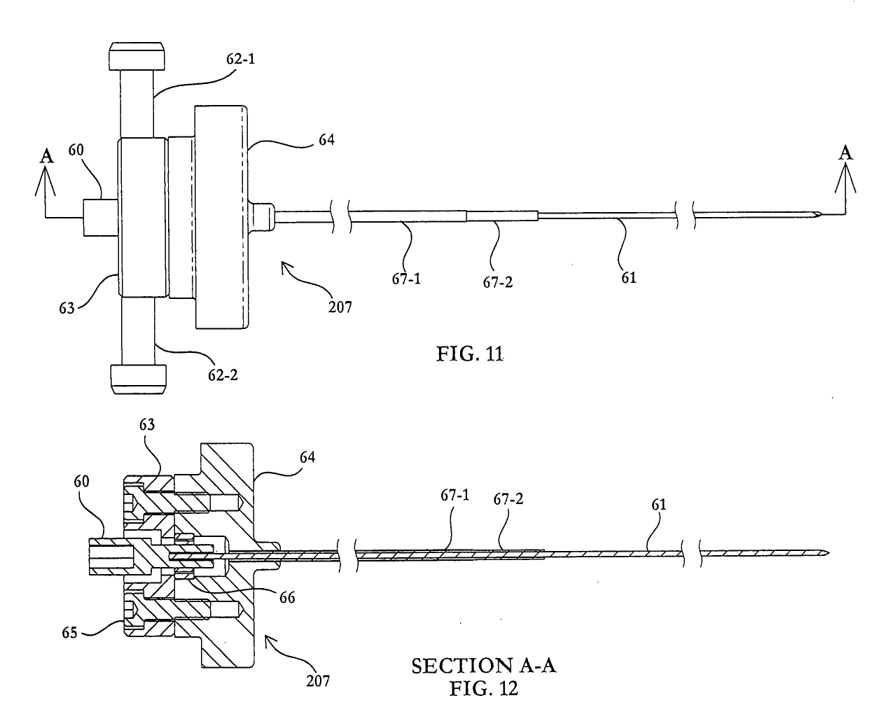

FIGS. 8-12 illustrate a disposable drilling assembly 201 and its components, in accordance with the invention. The disposable drilling assembly 201 generally includes a clear motor guide tube 40, a deployment/retraction lever 41 (which is also referred to herein as an actuation lever), an indexing post 42, a motor attachment component (coupler) 60 of drill cable assembly 207, a guide tube 43, a hook 50 of drill guide assembly 206 (also called hook assembly), and a flexible drill cable 61 of drill cable assembly 207 that extends through the drill guide assembly 206. In one aspect of the invention, the drill cable 61 is moveable or slideable relative to the hook 50. In addition, the hook 50 is arcuate and retractable by means of lever 41 which is connected to hub 302 which is connected to tubes 51-1, 51-2 and 51-3. Tube 51-3 connects to a flexible spring 52 that then connects to the hook 50. Connecting the tube 51-3 to the hook 50 with a spring provides the means for the hook to retract within a channel in tip 53 of the disposable drill assembly 201. The fully extended position of the hook 50 can be optimized to provide the best drilling angle relative to the bone, such as between about 80 degrees and about 90 degrees relative to the axis of the guide tube 43. In one exemplary embodiment, the angle formed between the hook 50 and the axis of the guide tube 43 is about 86.5 degrees.

The guide tube 43 allows the drill cable 61 to be deployed inside the limited space of the inner cavity 107 of an intramedullary rod 106 during a surgical procedure. A large bend radius for the drill cable 61 which is defined by the bend radius of hook 50 can help to minimize the stresses on the drill cable.

The drill cable 61 is connected to a drill motor coupler 60 that is pressed into bearing 66, which in turn is held in place between bearing blocks 63 and 64. The bearing housing 63 further has pins 62-1 and 62-2 that interface with the motor guide tube 40 and prevent rotation of the bearing housing assembly within the guide tube. Tubes 67-1 and 67-2 connect to bearing block 64 and help stabilize and guide the drill cable 61.

Disposable drilling assembly 201 is intended to be a single-use component that can be used for one surgical procedure, and then disposed of after the procedure is complete. The drill motor 32 of drill motor assembly 203 is attachable to the disposable drilling assembly 201 by aligning the pins 36 of drill motor 32 to the guide channels 44 of the guide tube 40. The drill motor 32 is then slid into the motor guide tube 40 and “twisted” so that the pins 36 follow channels 44 into their longitudinal portion 46. The illustrated embodiments incorporate two of such guide channels 44 and longitudinal portions 46 directly opposed to one another. The guide channels 44 are slots in the motor guide tube 40 that are open at the proximal end 45 of the device to accept pins 36 (refer to FIG. 7) or other locator devices that extend from the sides of the housing of the drill motor 32. The illustrated guide channels 44 extend at an angle from their open end at the proximal end 45 in a somewhat circumferential direction, then turn and extend in a longitudinal direction along a portion of the length of the motor guide tube 40. This longitudinal portion 46 of each of the guide channels 44 is preferably parallel with the longitudinal axis of the motor guide tube 40. In this way, the drill motor 32 can be advanced linearly along the length of the inside of the motor guide tube 40 when the drilling operation is occurring.

Although the motor guide tube 40 is shown with two guide channels 44, it is contemplated that more or less than two guide channels are provided, and/or that the guide channels are configured differently. In any case, the guide channels 44 are preferably sized and configured to allow secure attachment of the drill motor 32 to the guide tube 44 while allowing smooth movement of the drill motor relative to the guide tube. Thus, the guide channels 44 can be larger, smaller, or differently shaped than is shown in order to accommodate the size and shape of the pins 36 or other features that extend from the sides of the drill motor 32.

The housing of the drill motor 32 has guide channels 230 that are similar to the guide channels 44 of the motor guide tube 40. During connection of drill motor assembly 203 to disposable drill assembly 201, the drill cable 61 is fully retracted so that the bearing blocks 63 and 64 are towards the proximal end 45 of the guide tube 44. Next, at the same time as the drill motor 32 and its pins 36 are engaging the motor guide tube 40 and the channels 44, the drill motor guide channels 230 are engaging the pins 62-1 and 62-2 of drill cable assembly 207, and also at the same time the drive shaft 70 of drill motor 32 engages the drill cable coupler 60 of drill cable assembly 207. Pins 62-1 and 62-2 also pass through the guide channels 44 in separate slots 236-1 and 236-2, and prevent the bearing housings 63 and 64 from rotating within the guide tube 40.

With the drill motor 32 engaged into the longitudinal portion 46 of the guide channels 44 and connected to drill cable assembly 207, the next step in attaching the drill motor assembly 203 to the disposable drilling assembly 201 is to attach the motor guide-tube cap 33. This step is accomplished by aligning two diametrically opposed pins 37 of the motor guide-tube cap 33 to the guide channels 44. The cap 33 is then pushed onto proximal end 45 of the guide tube 40 with a “twisting” motion so that the pins 37 follow the guide channels 44. When the proximal end 45 bottoms out against the inside surface 38 of the motor guide-tube cap 33 the cap is fully engaged. Now, the outer push/pull guide 31, which is fastened to end cap 33, provides a conduit through which the linear stage 10 can advance and retract inner cable 30 which is connected to drill motor 32 that is now inside the guide tube 40 and engaged with the drill cable 61 via the drill motor coupler 60.

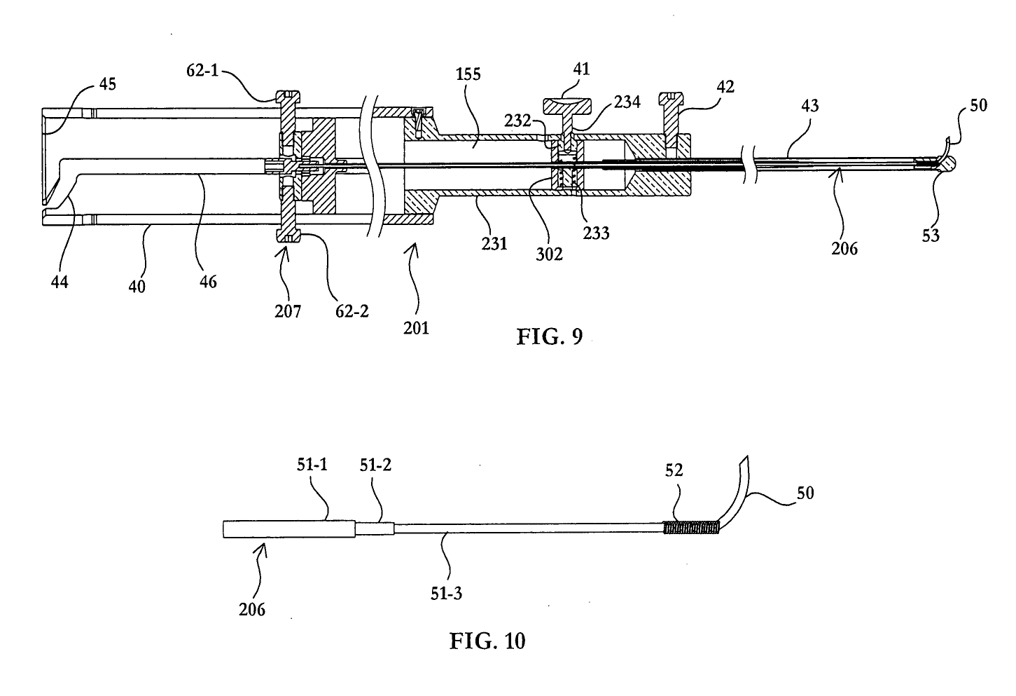

The disposable drill assembly 201 includes a distance limiter 231 from which the lever 41 and indexing post 42 extend, as shown in FIGS. 8 and 9. This distance limiter 231 is a cylindrical portion with inside cylindrical cavity 155 and is adjacent to the motor guide tube 40. The deployment/retraction lever 41 is used for deployment and retraction of the hook 50, which is at the distal end of the disposable drilling assembly 201 and connected via tubes 51-1, 51-2 and 51-3 to the hub 302 that is inside the distance limiter 231.

Referring to FIGS. 8 and 9, the lever 41 is threaded into a plunger 232 having a portion with a smaller diameter that extends past the outside of the distance limiter 231 and a portion having a larger diameter that does not extend through the wall or outside the limiter. The plunger 232 moves within the hub 302 that fits and slides along the inside walls of cavity 155 of limiter 231. The smaller diameter portion of the plunger 232 is larger than the diameter of the shaft 234 of lever 41. A spring 233 within hub 302 aids in the movement of the lever 41 connected to plunger 232 toward and away from the outer surface of the limiter 231. This motion allows the smaller diameter portion of the plunger 232 to extend through the external wall of the limiter when the lever 41 is released, or to only have the lever shaft 234 (that has an even smaller diameter) extend through the external wall of the limiter when the lever 41 is depressed.

These features just described now provide a means to lock the lever 41 in place at specific positions along the limiter 231 as well as the hook 50, since the hook is connected via different components to lever 41. This controlled deployment and retraction of the hook 50 is accomplished because of slot 301 with enlarged ends 300-1 and 300-2 in the distance limiter 231. The sizes of the slot 301 and the enlarged ends 300-1 and 300-2 are such that the lever 41 can freely extend through all of them. However, the smaller-diameter end of plunger 232 can only extend through the enlarged openings 300-1 and 300-2, i.e. its diameter is larger than the width of slot 301. With this design, the lever 41 must be depressed to freely deploy and retract the hook 50, and the lever can only fully extend outward when in the end positions 300-1 and 300-2. Furthermore, when lever 41 is fully extended it is locked in place since in this state plunger 232 is passing through one of the enlarged openings 300-1 and 300-2 and does not allow further movement along slot 301.

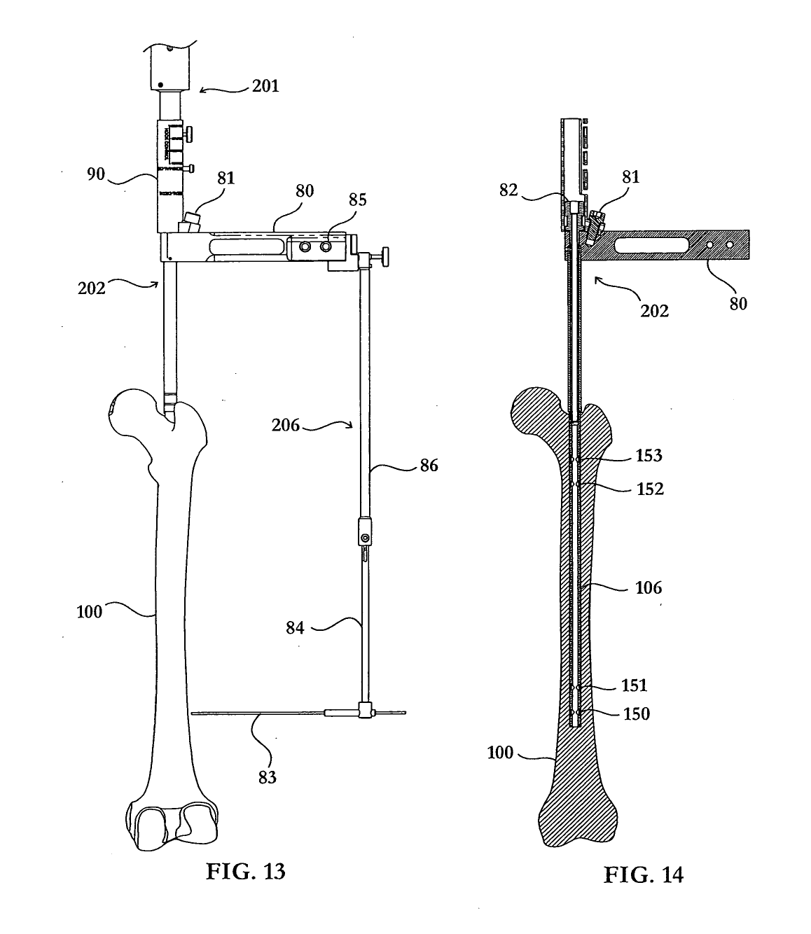

Referring now to FIG. 13 and associated cross-section view FIG. 14, the disposable drilling assembly 201 interfaces with a nail-interface assembly 202 through a jig interface 90 that attaches to a nail-attachment jig 80 with a bolt 81. The nail-interface assembly 202 is comprised of a nail-attachment jig 80 whose cannulated shaft portion connects to the intramedullary nail 106 using a cannulated retention bolt 82 that fits inside the cannulated shaft of jig 80. In the surgical procedure, the intramedullary nail 106 is delivered into the intramedullary canal 102 while connected to the nail-attachment jig 80. Other instruments and tools that would interface to the nail-attachment jig 80 to accomplish this delivery of the nail into the intramedullary canal 102 are common and known to those skilled in the art and not illustrated here. The intramedullary rod 106 includes a distal/distal hole 150 and a proximal/distal hole 151, along with holes at the proximal end of the rod 152 and 153. The nail interface assembly 202 may be saved for sterilization and reuse purposes after its initial use.

An incision targeting assembly is also illustrated in FIGS. 2 and 13 and connects to the extending arm of nail-attachment jig 80. It is comprised of a targeting pin 83 and a distance indicator 84. The incision targeting assembly 208 indicates to the surgeon as to where to make the incision to expose the bone where the drill cable 61 is expected to exit. The incision targeting assembly 208 can be reused, and therefore can be wrapped in an autoclave drape and sterilized after its use, possibly at the same time that other components from the procedure are being sterilized.

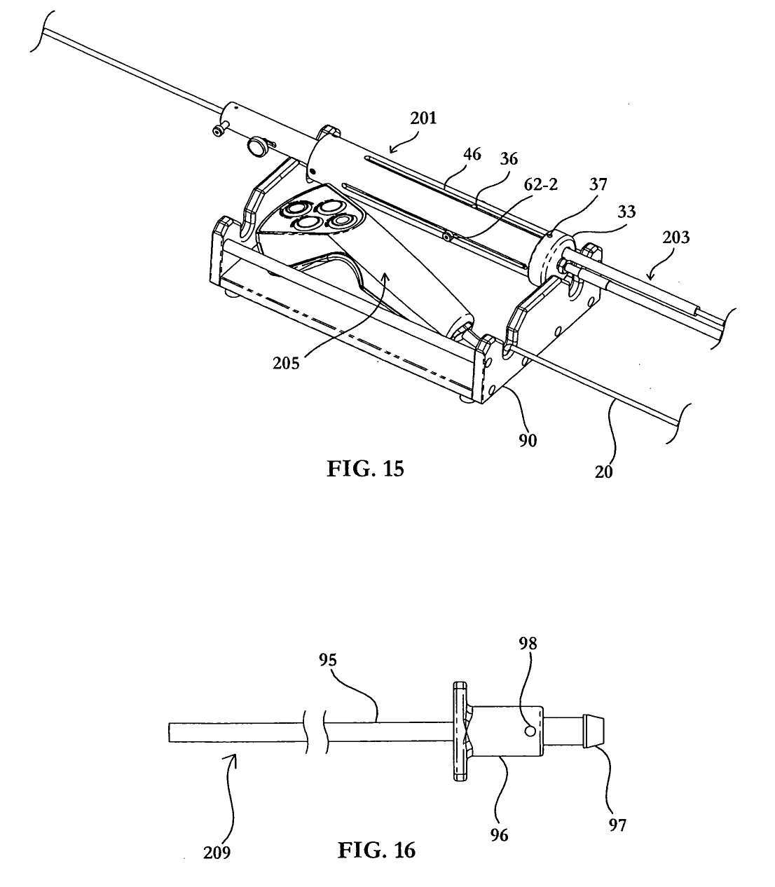

FIG. 15 shows a holster 90 which provides a means to store the disposable drilling assembly 201 and hand control assembly 205 in the sterile field; this helps prevent these devices from otherwise potentially falling to the ground and becoming unusable due to loss of sterility. FIG. 15 also shows the disposable drilling assembly 201 connected to the drill motor assembly 203.

FIG. 16 illustrates a suction tube assembly 209, which is used to clear the inside of the intramedullary rod 106 after the rod has been delivered into the intramedullary canal 102. A slender tube 95 of assembly 209 has an outer diameter small enough that it can pass down the inside (cannula) 154 of the intramedullary nail 106. A fitting 96 is attached to the proximal end of the tube and is shown with a barb interface 97 that can be attached to an appropriately sized flexible tube from an available vacuum system. An opening 98 through the fitting allows air to enter and bypass the slender tube 95 thereby providing a means to reduce the amount of suction provided to the slender tube 95. The surgeon can cover this opening 98 with their finger to cause suction to be provided only through tube 95. This port 98 thereby provides the surgeon a simple way to increase or decrease suction through tube 95. The suction tube assembly 209 is necessary because intramedullary nails are generally open on the distal end. As the intramedullary nail 106 is inserted into the intramedullary canal 102, material from within the intramedullary canal 102 can enter the inside of the nail 106 and prevent the disposable drilling assembly 201 from being properly positioned inside the intramedullary nail 106. The suction tube assembly 209 too can be sterilized with the other instrumentation from the intramedullary fixation or nailing procedure. The suction tube assembly 209 is just one example of providing a means to clear the inside of the nail 105. The assembly 209 can also be plunged in and out of the intramedullary nail 106 without using suction. This action tends to push any blockage into the tube 95 which can then be emptied.

In order to prepare the various components described above for use in a drilling operation, a number of exemplary steps can be performed, where it is understood that variations of the order of these steps are contemplated, along with the addition or deletion of steps or processes. Referring now to all the figures, in this exemplary process, the control box system 204 is placed on a secure table or Mayo stand just outside of the sterile field near the surgical location. Preferably, the positioning of this control box 204 will allow the drill motor assembly 203 to reach the intramedullary nail 106 within a range of four feet in both the horizontal and vertical directions. The total number of bends provided in the drill motor assembly 203 preferably does not exceed 360 degrees, although it is possible that the total number of bends can be larger than this. A power cord can then be plugged at one end into a standard outlet, such as a dedicated 120V outlet, and plugged in at the opposite end into the power entry module 3 at the rear of the control box assembly 204. After the power cord is attached, the on/off switch that is part of the power entry module 3 can be switched to the “on” position to prepare the control box assembly 204 for operation.

A number of drapes may be used in the procedure, although the exact use of such drapes can vary considerably. In one process, the hand control connector 26 is attached to the hand control port 1 on the front of the control box 204, and a drape can be slid over the hand control housing 21 and down the length of the hand controller assembly 205. Adhesive tape or another material or device can be used to secure the drape in this position. The draped hand control can now be placed on a table or in the holster 90 that can be on a table in the sterile field as illustrated in FIG. 15.

The drill motor assembly 203 can now be connected to the control box system 204. This is done in the following exemplary manner. The outer push/pull control box connection 34-2 can be connected to the push/pull outer fitting 4-2 of the control box system 204, and the inner push/pull cable connector 34-1 can be connected to the push/pull inner fitting 4-1 on the control box system 204.

The drill motor assembly 203 can then be connected to the disposable drilling assembly 201 by aligning the pins 36 of the drill motor 32 with the guide tube slots 44 of the motor guide tube 40. The drill motor 32 is then slid into the motor guide tube 40 and twisted along the channels 44 until the pins are positioned within the longitudinal portion 46 of the slots, as shown for example in FIG. 15. The motor guide-tube cap 33 can then be connected to the motor guide tube 40 by aligning the diametrically opposed pins 37 with the same slots 44 until the cap is fully seated, which is when the pins are aligned with the longitudinal portion 46 of the slots 44, as is illustrated in FIG. 15. The assembled drill system can then be placed on the holster 90 in the sterile field as shown in FIG. 15.

The next step to prepare for the surgical procedure is to attach the nail interface assembly 202 to the intramedullary nail or rod 106 using the cannulated bolt 82 as shown in cross-section view in FIG. 14. After the intramedullary rod 106 has been inserted into the femur of the patient, the arm 86 of the incision targeting assembly 206 can then be attached to the nail interface assembly 202 with bolt 85. The bolt can be tightened as desired to provide stability to the assembly. The distance indicator 84 is then adjusted relative to the arm 86 to an appropriate length that is dependent on the length of the intramedullary rod 106. A targeting pin 83 can then be inserted through the end of the distance indicator 84, where it will point to, for example, the distal/distal hole 150 of the intramedullary rod 106. An incision can then be made in the patient, to expose the area where the drilling cable 61 will exit the bone for the distal/distal hole 150 and the proximal/distal hole 151. For one example, the incision can be 4 cm in length and can start at a distance of 1 cm distal to the targeting pin so that the area where the drilling cable 61 will exit becomes exposed. After the incision is made, the incision targeting assembly 206 can be removed. The tissue of the incision area can be separated using standard retractors, such as Hohmann retractors, to expose the drilling exit bone surface.

Next, the jig interface 90 can be attached to the nail-interface assembly 202 using bolt 81. Bolt 81 can be tightened using standard bolt tightening techniques until a desired tightness is achieved.

A suction tube assembly 209 or a vacuum tube can then be inserted into the inner channel 154 of the intramedullary rod 106 and attached to a vacuum source to extract extraneous fluids and debris from the intramedullary channel or cavity. The suction tube assembly 209 can then be removed from the intramedullary nail 106.

The disposable drill assembly 201 can then be inserted into the jig interface 90, which guides the tip of the disposable drill assembly where the hook 50 resides into the cannulated connection bolt 82 which further guides the tip into the bore 154 of the intramedullary nail 106.

When the disposable drill assembly 201 has been fully inserted as shown in FIG. 13, the indexing post 42 and deployment/retraction lever 41 can be rotated so that the indexing post engages transverse slot 160, which can be labeled “distal/distal”, for example, on the jig interface 90. This slot 160 is seen in FIG. 18. The devices are now positioned for drilling through a hole of the intramedullary rod 106 and the adjacent bone. In particular, the devices are ready for passing the hook through the distal/distal hole 150 of the rod 106 and drilling into the adjacent bone structure.

Referring in particular to FIG. 9, in order to deploy the hook 50, plunger 232 connected to the deployment/retraction lever 41 is disengaged from distance limiter 231 by depressing the lever 41. This allows the hook assembly 206 that is connected to the hub 302 to be slid distally. When the shaft 234 of lever 41 has reached its maximum travel distance along the limiter 231 defined by the open area 300-2, lever 41 will spring back to its original height re-engaging the plunger 232 into the hole 300-2 of the distance limiter 231, thereby locking the hook assembly 206 into position.

When the hook 50 is fully deployed, the drilling procedure can begin. In order to start this drilling, the start/stop button 22 on the hand controller 21 of the hand control assembly can be pressed or activated. The bone area that has been exposed is then observed closely to watch for the drill cable 61 to emerge through the drilling surface (i.e., the outer surface of the bone). When the drill cable 61 is visible, the start/stop button 22 can again be pressed or activated to stop the drilling operation. It is preferable that the drill bit 61 is not allowed to extend more than 1 cm past the outer surface of the bone. If necessary, the jog forward button 24 which advances the drill cable 61 without rotating it can be pressed to extend the drill cable further out of the bone until it is visible.

Referring to FIG. 18, a chase-back pin 400 can then be inserted into the hole that was made in the bone by the drill cable 61, while using the jog back button 25 on the hand controller 21. Once the chase-back pin 400 has been engaged within the exit hole (also called a pilot hole) in the bone, the full retract button 23 of the hand controller assembly 205 can be pressed or activated to prepare for removal of the disposable drilling assembly 201. The chase-back pin 400 can take many forms and may preferably have a substantially smooth surface, but can also have threads or other surface features, as well as different tip designs such as blunt, rounded or trocar styles.

To retract the hook 50, the plunger 232 is disengaged from the distance limiter 231 by depressing deployment/retraction lever 41 and sliding it proximally along slot 301 (seen in FIG. 8) until the hook is fully retracted, which is when the lever 41 and plunger 232 reach the enlarged slot 300-1. The lever 41 is released and the plunger 232 re-engages the distance limiter 231. After retraction of the hook 22, the disposable drilling assembly 201 can be rotated within the jig interface 90 and pulled back by a sufficient distance so that the distal/distal locking hole 150 is clear.

A cannulated drill bit 401 can then be slid over the chase-back pin 400 and used with a standard surgical drill to enlarge the pilot hole through the first cortical wall that was made by the drill cable 61. Once this is accomplished, the cannulated drill bit 401 can be advanced through the hole of the nail. In order to confirm proper drilling with the cannulated drill bit 401, the disposable drilling assembly 201 can be slid back down until the user can feel it touch the cannulated drill bit 401. The disposable drilling assembly 201 can then be retracted again to its previous position. Drilling can now proceed, and the cannulated drill bit 401 used to penetrate and drill through the second far-side cortical wall of the bone. The cannulated drill bit 401 and chase-back pin 400 are then removed. A bone screw can now be implanted through the intramedullary nail 106 in a manner commonly done and familiar to surgeons. The bone screw is driven into this hole in the bone and through the nail, thereby locking the nail to the bone which helps prevent the nail from moving or rotating relative to the bone. An example of an alternative approach is to remove the cannulated drill bit 401 and chase-back pin 400 after drilling through the near cortical wall. Then a same-sized, standard, non-cannulated surgical drill bit is slipped through the hole that was just made and through the nail hole. Then the drill bit is used to drill through the far cortex wall using a standard surgical drill.

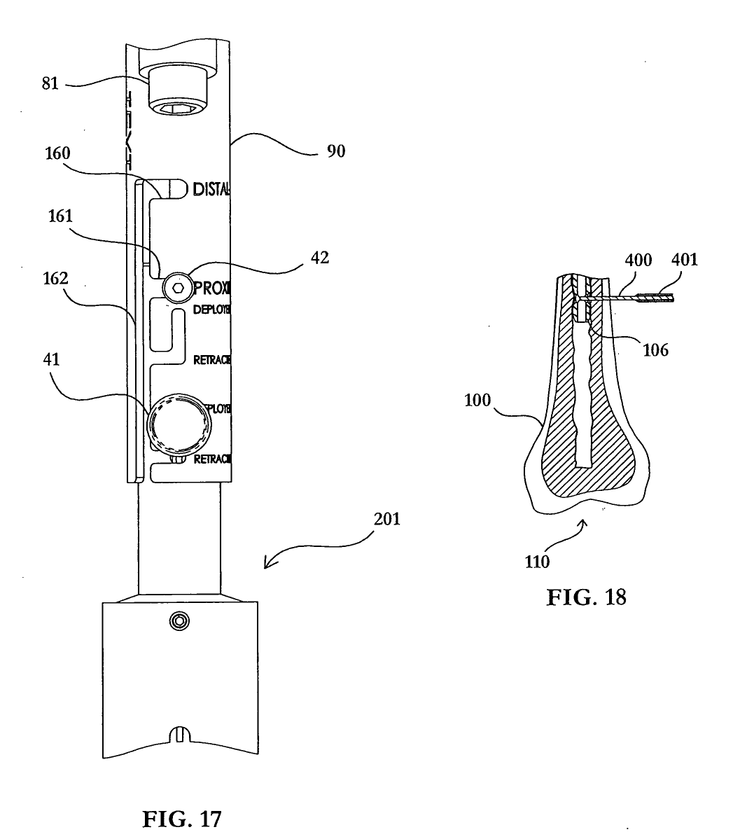

The next step in the process is to drill the proximal/distal hole 151 and insert a screw into this hole for additional fixation of the intramedullary rod 106 to the bone at the proximal/distal location. Referring in particular to FIGS. 9 and 17, for this procedure the disposable drilling assembly 201 is rotated such that shaft 234 of lever 41 and the indexing post 42 move into the long slot 162 of jig interface 90. Now the disposable drilling assembly 201 can be slid proximally and rotated back into a position where the indexing post is in slot 161, which can be labeled “proximal/distal”, for example, on the jig interface 90. FIG. 17 shows this configuration of the disposable drilling assembly 201 relative to the jig interface 90. The drilling procedure outlined previously for the drilling procedure can be repeated for the proximal/distal hole 151. When the proximal/distal locking screw has been inserted in its desired position, the disposable drilling assembly 201 can be removed, disconnected, and discarded.

After the procedure is complete, the remaining components may have their drapes removed and discarded, and then the components themselves should be cleaned and prepared for the appropriate sterilization method. The drill motor assembly 203 can then be disconnected from the control box system 204 and its wires can be managed in an appropriate manner. In one exemplary procedure, the drill motor assembly 203 can be coiled into three loops so that the terminated ends diametrically opposed to each other. Autoclave-rated straps can then be used to fasten the three loops together at the two ends. If the drill motor is to be placed into the autoclave, it should be verified that the bend radius of the cable is not too tight. The surfaces of the control box 9 and associated power cord can then be cleaned with an appropriate cleaning product.

The present invention has now been described with reference to several embodiments thereof. One exemplary embodiment that was described in detail included a drill cable 61 structured to make a small pilot hole through the bone, which then identifies to the surgeon where to implant the locking screws. The invention of making a pilot hole through the bone starting from the inside cannula 154 of an intramedullary nail 106 can be accomplished with other means and are included in the spirit of this invention. Also the invention was described in the context of locking an intramedullary nail in a femur, but applies to locking any cannulated implant in any bone as will be appreciated by those skilled in the art.

The entire disclosure of any patent or patent application identified herein is hereby incorporated by reference. The foregoing detailed description and examples have been given for clarity of understanding only. No unnecessary limitations are to be understood therefrom. It will be apparent to those skilled in the art that many changes can be made in the embodiments described without departing from the scope of the invention. Thus, the scope of the present invention should not be limited to the structures described herein.

What is claimed is:

1. A drill assembly comprising:
a motor guide tube;
an adaptor extending from a distal end of the motor guide tube;
an elongate drill guide tube extending from a distal end of the adaptor;
a retractable guide tube including an arcuate distal end slideably disposed within a channel of the drill guide tube;
an actuation lever operably coupled to the retractable guide tube and extending from an outer surface of the adaptor;
a flexible drill cable slideably disposed within the retractable guide tube, wherein a distal end of the flexible drill cable is structured to be advanced through the arcuate distal end of the retractable guide tube;
a drill motor operably coupled to the flexible drill cable; and
a push-pull cable extending between the drill motor at a distal end and a liner stage motor inside a control box at a proximal end, the linear stage motor operable to advance and retract the push-pull cable and the drill motor.

2. The drill assembly of claim 1, further comprising a jig interface slideably engageable with the actuation lever.

3. The drill assembly of claim 2, wherein the arcuate distal end of the retractable guide tube is slideable between a retracted position wherein the arcuate distal end is contained within the channel of the drill guide tube and an extended position wherein the arcuate distal end extends from an opening in a distal end of the drill guide tube.

4. The drill assembly of claim 3, wherein the arcuate distal end forms an angle with a longitudinal axis of the drill guide tube between about 80 degrees and about 90 degrees when the arcuate distal end is in the extended position.

5. The drill assembly of claim 3, wherein the actuation lever is slideable within an actuation lever channel of the jig interface to control movement of the arcuate distal end of the retractable guide tube between the retracted position and the extended position.

6. The drill assembly of claim 5, further comprising an indexing post extending from the outer surface of the adaptor.

7. The drill assembly of claim 6, further comprising an intramedullary rod having a bore extending therein that is structured to slideably receive the drill guide tube.

8. The drill assembly of claim 7, wherein the jig interface includes a first slot engageable with the indexing post to position the opening in the distal end of the drill guide tube adjacent to a first distal aperture in the intramedullary rod.

9. The drill assembly of claim 8, wherein the jig interface includes a second slot engageable with the indexing post to position the opening in the distal end of the drill guide tube adjacent to a second distal aperture in the intramedullary rod.

10. The drill assembly of claim 1, wherein the drill motor is enclosed by a motor housing, the motor housing including at least one engagement member for engagement with the motor guide tube.

11. The drill assembly of claim 10, wherein the motor guide tube includes a slot extending from a proximal end of the guide tube toward the distal end of the guide tube, and wherein the at least one engagement member comprises a pin extending from an outer surface of the motor housing for engagement with the slot of the motor guide tube.

12. The drill assembly of claim 1, wherein the flexible drill cable is formed from Nitinol.

13. The drill assembly of claim 1, wherein the drill motor is operable to rotate the flexible drill cable.

14. The drill assembly of claim 1, wherein the flexible drill cable further comprises an energy delivery means for delivering ablation energy to the distal end of the drill cable.

15. The drill assembly of claim 1, wherein a proximal end of the drill cable is supported by at least one drill cable guide tube.

16. The drill assembly of claim 1, further comprising a spring member disposed between the arcuate distal end and an elongate main body of the retractable guide tube that is structured to allow the arcuate distal end to retract within the channel of the drill guide tube.

17. The drill assembly of claim 16, wherein a proximal end of the retractable guide tube includes a plurality of substantially concentric tubes operably coupled to the actuation lever that are structured to allow retraction and extension of the arcuate distal end.

18. A method of forming a pilot hole through an intramedullary rod positioned within a bone comprising:
inserting an intramedullary rod into a cavity of a bone;
providing a drilling assembly;
coupling the drilling assembly to the intramedullary rod such that a drill guide tube is disposed within a bore of the intramedullary rod;
positioning a distal end of the drill guide tube adjacent to a distal aperture of the intramedullary rod;
advancing an arcuate distal end of a retractable guide tube through an opening in the distal end of the drill guide tube; and
forming a pilot hole by advancing a drill cable through the arcuate distal end of the retractable guide tube and into the bone, wherein the drill cable is advanced by a push-pull cable extending between the drill cable at a distal end and a linear stage motor within a control box at a proximal end.

19. The method of claim 18, wherein the drilling assembly is coupled to the intramedullary rod with a rod interface assembly.

20. The method of claim 18, wherein the arcuate distal end forms an angle with a longitudinal axis of the drill guide tube between about 80 degrees and about 90 degrees when the arcuate distal end is advanced through the opening in the drill guide tube.

21. The method of claim 18, wherein the drilling assembly includes a drill motor operably coupled to the drill cable.

22. The method of claim 21, wherein the drill motor is operable to rotate the drill cable.

23. The method of claim 18, further comprising the steps of retracting the drill cable into the arcuate distal end of the retractable guide tube and inserting a chase back pin into the pilot hole.

24. The method of claim 18, further comprising the steps of positioning the opening in the distal end of the drill guide tube adjacent to a second distal aperture of the intramedullary rod and drilling a second pilot hole into the bone.

25. The method of claim 18, wherein the drilling assembly further comprises an actuation lever operably coupled to the retractable guide tube, wherein the actuation lever is operable to control movement of the arcuate distal end of the retractable guide tube between a retracted position and an extended position.

26. A system for forming a pilot hole through an intramedullary rod positioned within a cavity of a bone comprising:
a drilling means;
means for coupling the drilling means to the intramedullary rod such that a drill guide tube of the drilling means is disposed within a bore of the intramedullary rod;
means for positioning a distal end of the drill guide tube adjacent to a distal aperture of the intramedullary rod;
means for advancing an arcuate distal end of a retractable guide tube through an opening in the distal end of the drill guide tube; and
means for forming a pilot hole by advancing a drill cable through the arcuate distal end of the retractable guide tube and into the bone, wherein the drill cable is advanced by a push-pull cable extending between the drill cable at a distal end and a linear stage motor within a control box at a proximal end.

27. The system of claim 26, wherein the arcuate distal end of the retractable guide tube forms an angle with a longitudinal axis of the drill guide tube between about 80 degrees and about 90 degrees when the arcuate distal end is advanced through the opening in the drill guide tube.

28. The system of claim 26, wherein the drilling means includes a drill motor operably coupled to the drill cable.

29. The system of claim 28, wherein the drill motor is operable to rotate the drill cable.

30. The system of claim 26, wherein the means for advancing the arcuate distal end comprises an actuation lever operably coupled to the retractable guide tube, wherein the actuation lever is operable to control movement of the arcuate distal end of the retractable guide tube between a retracted position and an extended position.
